(19) United States
(12) Patent Application Publication
(10) Pub. No.: US 2018/0340208 A1
(43) Pub. Date: Nov. 29, 2018

(54) COMPOSITIONS AND METHODS FOR ABSOLUTE QUANTIFICATION OF PROTEINS

(71) Applicant: WOODS HOLE OCEANOGRAPHIC INSTITUTION, WOODS HOLE, MA (US)

(72) Inventors: MAKOTO SAITO, WOODS HOLE, MA (US); MATTHEW R. MCILVIN, WOODS HOLE, MA (US)

(73) Assignee: WOODS HOLE OCEANOGRAPHIC INSTITUTION, WOODS HOLE, MA (US)

(21) Appl. No.: 15/768,421

(22) PCT Filed: Oct. 14, 2016
(86) PCT No.: PCT/US16/57160

§ 371 Date: Apr. 13, 2018

Related U.S. Application Data
(60) Provisional application No. 62/242,137, filed on Oct. 15, 2015.

(51) Int. Cl. C12Q 1/37; C12P 21/02; C07H 21/04; C07K 14/00; C12N 5/07

(52) U.S. Cl. CPC C12Q 1/37; C12P 21/02; C12N 5/06; C07K 14/00; C07H 21/04

(57) ABSTRACT

Described herein is a method for producing and calibrating isotopically labeled peptides for use as standards for a variety of analytical and qualitative purposes. An expression construct, encoding a superprotein comprised of concatenated peptides, proteolysis sensitive sites, a detectable moiety, and optionally, a purification tag, is provided. The quantity of the superprotein and/or peptide is determined using the detectable moiety. The superprotein is subjected to proteolytic digestion and the peptides are isotopically labeled.

### CROSS REFERENCE TO RELATED APPLICATION

This application claims priority to and the benefit of U.S. Provisional Application 62/242,137 entitled “Compositions and Methods for Absolute Quantification of Proteins” filed on Oct. 15, 2015, which is incorporated herein by reference and without disclaimer.

### BACKGROUND OF THE INVENTION

As the technical capabilities for mass spectrometry-based protein measurements have improved, interest in determining absolute concentrations of individual proteins has increased. To enable absolute quantitation, calibration to an added isotopically labeled internal standard is the preferred approach, with a “heavy” labeled peptide standards corresponding to each “light” naturally occurring peptide targeted within the sample. Labeled peptides can be produced by various peptide synthesis methods or by heterologous expression systems such as within E. coli bacteria. In the former case, synthesized peptide standards are generally isotopically labelled and extensively purified in order to precisely determine their concentration. Such peptides are then provided in pre-calibrated, lyophilized aliquots which are expensive, have limited freezer shelf life, and could be subject to losses during solubilization of lyophilized aliquots and during freeze/thaw cycles. Moreover, synthesized peptides do not typically provide a means for users to independently verify accuracy.

As mentioned, quantitation accuracy remains unreliable due to difficulty in re-dissolving the purified peptides as a result of their chemical characteristics, such as hydrophobicity, peptide size, etc. As a result of using the current peptide standards, there are problems with reproducibility and accuracy, which can lead to significant issues in the determination of protein concentrations because inaccuracies in protein standard concentrations result in inaccuracies in the absolute measurement of protein concentration.

The production of peptide standards for use in analytical methods has also been expensive. For example, a small library of 20 peptide standards produced by a biotechnology company easily costs $500 to $1,000 for each standard and needs to be replaced approximately every two years for reliable protein analysis. Not only is this cost a major burden on individual research laboratory ($50,000 to $100,000 per year in replacement costs), but further peptide library scale-up to 500 to 1,000 peptide standards, while technically possible, is economically out of reach for these non-biomedical research groups and individual laboratories.

One particular field which would greatly benefit from large-scale absolute protein quantification is environmental research and monitoring. For the development of protein measurements, such as using targeted metaproteomic methods, to detect changes in natural environments, such as in the oceans or lakes, where there are many microorganisms that contribute to ecosystem structure and biogeochemical function, there is a need for the production of many peptides standards that can be regularly produced and shared among investigators globally to provide successful intercalibration. The combination of smaller research funds available to environmental researchers and the much larger number of potential proteins targets makes the development of a high-throughput heterologous peptide standard production approach very appealing. Furthermore, the essential need for long-term accuracy in absolute units is especially apparent in dynamic sampling environments since comparison to a control reference standard is not possible where environmental samples comprise a community of organisms that change across space and time.

Thus, high quality standards with verifiable accuracy and high-throughput production enabling improved absolute protein quantitation are needed.

### SUMMARY OF THE INVENTION

The present invention is related to the field of protein quantification particularly on an absolute scale. As described below, the present invention features carousel peptides useful as internal standards for absolute quantification of analyte proteins, as well as methods for generating and using the carousel peptides. The application of targeted metaproteomics calibrated by carousel peptides is particularly well suited to the assessment and monitoring of the microbial and phytoplankton component of complex ecosystems for environmental assessment.

In one aspect, the invention provides a superprotein useful for generating an internal standard for quantifying an analyte protein, the superprotein including a plurality of carousel peptides consecutively linked by a protease cleavable site to form a chain of peptides, where each carousel peptide is a fragment of an analyte protein pre-identified as a product of protease cleavage of the analyte protein; and a detectable protein fused to the chain of peptides.

In another aspect, the invention provides a composition for generating an internal standard for quantifying an analyte protein, the composition containing a superprotein useful for generating an internal standard for quantifying an analyte protein, that includes a plurality of carousel peptides consecutively linked by a protease cleavable site to form a chain of peptides, where each carousel peptide is a fragment of an analyte protein pre-identified as a product of protease cleavage of the analyte protein and a detectable moiety fused to the chain of peptides; an isotopic label; and a protease.

In another aspect, the invention provides an isolated polynucleotide encoding the superprotein according to any aspect delineated herein.

In another aspect, the invention provides an expression vector containing a polynucleotide encoding a superprotein useful for generating an internal standard for quantifying an analyte protein, that includes a plurality of carousel peptides consecutively linked by a protease cleavable site to form a chain of peptides, where each carousel peptide is a fragment of an analyte protein pre-identified as a product of protease cleavage of the analyte protein; and a detectable protein fused to the chain of peptides.

In another aspect, the invention provides a host cell for expressing a superprotein useful for generating an internal standard for quantifying an analyte protein, that contains the isolated polynucleotide or the expression vector according to any aspect delineated herein.

In another aspect, the invention provides a kit containing the expression vector according to any aspect delineated herein.

In another aspect, the invention provides a method for generating a superprotein useful for quantifying an analyte protein, the method involving culturing a host cell in a medium, where the host cell heterologously expresses a superprotein useful for generating an internal standard for quantifying an analyte protein, that includes a plurality of carousel peptides consecutively linked by a protease cleavable site to form a chain of peptides, where each carousel peptide is a fragment of an analyte protein pre-identified as a product of protease cleavage of the analyte protein; and a detectable protein fused to the chain of peptides; and isolating the superprotein.

In another aspect, the invention provides a method for generating an internal standard useful for quantifying an analyte protein, the method involving culturing a host cell in a medium, where the host cell heterologously expresses a superprotein useful for generating an internal standard for quantifying an analyte protein, that includes a plurality of carousel peptides consecutively linked by a protease cleavable site to form a chain of peptides, where each carousel peptide is a fragment of an analyte protein pre-identified as a product of protease cleavage of the analyte protein; and a detectable protein fused to the chain of peptides; isolating the superprotein; measuring fluorescence or activity of the isolated superprotein relative to a fluorescence or activity standard to determine an amount of the superprotein; and contacting the isolated superprotein with a protease, thereby cleaving the superprotein; contacting the isolated superprotein and/or cleaved superprotein with an isotopic label, thereby generating a carousel peptide useful as an internal standard for quantifying an analyte protein.

In another aspect, the invention provides a composition for absolute quantification of an analyte protein, that contains an amount of a carousel peptide, an amount of an isolated superprotein, or an amount of a cleaved superprotein, where the carousel peptide, isolated superprotein, or cleaved superprotein is generated according to the method of any aspect delineated herein.

In another aspect, the invention provides a method for absolute quantification of a polypeptide by mass spectrometry, the method involving obtaining a mass spectra of the composition of any aspect delineated herein.

In various embodiments of any aspect delineated herein, a purification tag is fused to a 3′ end or 5′ end of the superprotein. In certain embodiments, the purification tag is a histidine tag, a biotin tag, myc tag, a hemagglutinin (HA) tag, or a FLAG tag.

In various embodiments of any aspect delineated herein, the superprotein includes at least 2, at least 3, at least 4, at least 5, at least 10, at least 20, at least 30, at least 40, at least 50, at least 60, at least 70, at least 80, or at least 100 carousel peptides.

In various embodiments of any aspect delineated herein, the carousel peptides are fragments of at least 2, at least 3, at least 4, at least 5, at least 10, at least 20, at least 30, at least 40, at least 50, at least 60, at least 70, at least 80, or at least 100 different analyte proteins.

In various embodiments of any aspect delineated herein, the analyte protein is selected from one or more proteins involved in the ocean ecosystem (see, e.g., Table 2).

In various embodiments of any aspect delineated herein, the detectable protein or detectable moiety is a fluorescent protein or an enzyme (e.g., that produces a detectable signal when contacted with a substrate). In particular embodiments, the fluorescent protein is an enhanced green fluorescent protein (eGFP), red fluorescent protein (RFP), far-red fluorescent protein, blue fluorescent protein (BFP), cyan fluorescent protein (CFP), yellow fluorescent protein (YFP), or orange fluorescent protein.

In various embodiments of any aspect delineated herein, the protease is trypsin, chymotrypsin, thermolysin, pepsin, a serine protease, a cysteine protease, a metalloprotease, Lys-C, Lys-N, Asp-N, Glu-C, or Arg-C.

In various embodiments of any aspect delineated herein, the carousel peptides are interspaced with spacers (see, e.g., Table 1).

In various embodiments of any aspect delineated herein, the isotopic label is selected from ¹³C, ¹⁵N, ¹⁸O, ²H, ³⁴S, ⁷⁴Se, ⁷⁶Se, ⁷⁸Se, and ⁸²Se.

In various embodiments of any aspect delineated herein, the isolated superprotein and/or cleaved superprotein includes a detectable protein, or a fragment thereof.

In various embodiments of any aspect delineated herein, the expression vector is an overexpression vector.

In various embodiments of any aspect delineated herein, the host cell is a bacterial, yeast, or mammalian cell. In various embodiments of any aspect delineated herein, the host cell contains the isolated nucleotide or expression vector of any aspect delineated herein.

In various embodiments of any aspect delineated herein, the composition further includes an analyte protein to be quantified, or a fragment thereof.

In various embodiments of any aspect delineated herein, the kit further contains at least one reagent selected from one or more of a protease, an isotopic label, a fluorescence standard, or activity standard.

In various embodiments of any aspect delineated herein, the medium contains an isotopic label (e.g., ¹³C, ¹⁵N, ¹⁸O, ²H, ³⁴Se, ⁷⁴Se, ⁷⁶Se, or ⁸²Se).

In various embodiments of any aspect delineated herein, the method further involves measuring fluorescence or activity of the isolated superprotein relative to a fluorescence or activity standard to determine an amount of the superprotein.

In various embodiments of any aspect delineated herein, the method further involves contacting the isolated superprotein with a protease, thereby generating a carousel peptide useful as an internal standard for quantifying an analyte protein.

In various embodiments of any aspect delineated herein, the step of contacting the isolated superprotein with a protease, thereby cleaving the superprotein and the step of contacting the isolated superprotein and/or cleaved superprotein with an isotopic label, thereby generating a carousel peptide useful as an internal standard for quantifying an analyte protein are performed substantially simultaneously.

In various embodiments of any aspect delineated herein, the step of contacting the isolated superprotein with a protease, thereby cleaving the superprotein is performed subsequent to the step of measuring fluorescence or activity of the isolated superprotein relative to a fluorescence or activity standard to determine an amount of the superprotein.

In various embodiments of any aspect delineated herein, the method further involves measuring fluorescence or activity of the isolated superprotein contacted with the protease relative to a fluorescence or activity standard; and comparing the fluorescence or activity measured in the step of measuring fluorescence or activity of the isolated superprotein relative to a fluorescence or activity standard to determine an amount of the superprotein and the step of measuring fluorescence or activity of the isolated superprotein contacted with the protease relative to a fluorescence or activity standard to determine a cleavage efficiency.

In various embodiments of any aspect delineated herein, the isolating step involves lysing the host cell to obtain a lysate containing the superprotein, and isolating the superprotein by affinity chromatography.

In various embodiments of any aspect delineated herein, the amounts of one or more of the carousel peptide, isolated superprotein, or cleaved superprotein is known.

In various embodiments of any aspect delineated herein, the method further involves measuring a mass spectral signal corresponding to one or more of the carousel peptide, the analyte protein, and the detectable protein.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, peptide (e.g., 2 or more amino acids linked by a peptide bond), or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “analyte protein” or “analyte” is meant any protein whose quantification (particularly, absolute quantification) is desired. Typically, the analyte protein is in a sample comprising a mixture of multiple proteins.

By “calibrate” is meant to correlate known amounts of an agent with readings or signals from an instrument analyzing the agent. Typically, varying known amounts of the agent are correlated with signals or readings from the instrument analyzing the various amounts of the agent to generate a “standard curve.” In some embodiments, various known amounts of labeled and unlabeled peptides (e.g., carousel peptides) are analyzed by mass spectrometry, and mass spectrometry signals are correlated with the amounts of peptides (or, relative amounts of the labeled and unlabeled peptides) to generate a standard curve. The standard curve generated may be used to calculate an absolute amount of an analyte protein of unknown amount analyzed together with a calibrated internal standard peptide (carousel peptide) corresponding to the analyte protein.

By “carousel peptide” is meant a peptide having the amino acid sequence of a fragment of an analyte protein obtained when the analyte protein is digested with a protease. When analyzed by mass spectrometry, the analyte protein fragment and the carousel peptide yield substantially identical characteristic mass spectral signals (i.e., “mass shift”), although the magnitude or height of the peaks may be different, depending on the quantities of the fragment and carousel peptide. An isotopically labeled carousel peptide will yield a mass spectral signal slightly shifted from an unlabeled carousel peptide or an unlabeled corresponding fragment of the analyte protein. By comparing the magnitude or height of peaks of the mass spectral signals, relative quantities of isotopically labeled and unlabeled peptides may be determined. Absolute quantities of analyte proteins may be determined using internal standards (such as carousel peptides of the invention) that have been calibrated or quantitated. In some embodiments, the absolute quantity of an analyte protein is determined by comparing the mass spectral signal of a fragment of an analyte protein with the mass spectral signal of a carousel peptide corresponding to the fragment (particularly, an isotopically labeled carousel peptide that has been calibrated) and deriving an initial concentration of the carousel peptide using a pre-determined standard curve to yield an absolute amount of the analyte protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence, or amount of the analyte to be detected.

By “detectable label” or “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. In some embodiments, the detectable moiety is a detectable protein. In particular embodiments, the detectable protein is a fluorescent protein (e.g., enhanced green fluorescent protein (eGFP)) or an enzyme.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The terms “protein,” “polypeptide,” and “peptide” are used interchangeably herein to include any molecule comprising a plurality of amino acid residues linked by peptide or amide bonds.

As used herein, “obtaining” as in “obtaining an agent” includes synthesizing, purchasing, or otherwise acquiring the agent.

By “protease” or “proteinase” is meant any enzyme that catalyzes proteolysis (i.e., hydrolysis of a peptide bond linking amino acid residues in a polypeptide). In some embodiments, a protease cleaves or digests a protein at a site on the protein (i.e., a “protease cleavable site”). Cleavage or digestion of the protein by a protease generates fragments of the protein. In particular embodiments, the protease is trypsin.

By “reduces” is meant a negative alteration of at least 10%, 25%, 50%, 75%, or 100%.

By “reference” is meant a standard or control condition. In some embodiments, fluorescence of a superprotein comprising a fluorescent protein is measured relative to a “fluorescence standard.” A fluorescence standard may be fluorescence of a fluorescent protein (e.g. eGFP) that has been calibrated. In some other embodiments, an activity of a superprotein comprising an enzyme is measured relative to an “activity standard.” An activity standard may be activity of an enzyme that has been calibrated (e.g., known amounts of enzyme correlated with an output activity level, such as substrate and/or product amounts, particularly those easily detectable by an assay).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “superprotein” is meant a fusion protein comprising a plurality of carousel peptides concatenated or linked consecutively by a protease cleavage site to form a chain of peptides. In particular embodiments, the superprotein comprises a detectable protein fused to the chain of peptides. In other embodiments, a purification tag is fused to a 3′ or 5′ end of the superprotein. In various embodiments described herein, cleavage of the superprotein by a protease (e.g, trypsin) yields carousel peptides useful as internal standards for absolute quantification of analyte proteins (particularly, quantification by mass spectrometric methods).

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 [mu]g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

A “vector” is a composition of matter that comprises an isolated polynucleotide and that may be used to deliver the isolated polynucleotide to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the expression vector is a plasmid (e.g., high expression plasmid). In particular embodiments, the host cell is a bacteria (e.g., E. coli).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic showing the methods for creating carousel peptides described herein for quantitative proteomics. FIG. 1A depicts the expression plasmid which comprises the carousel peptides (labeled Pep) interspaced with cleavage spacers (labeled S), a purification handle (labeled Tag), and a reporter. FIG. 1B shows the superprotein derived from the expression plasmid is calibrated, added to a sample to be quantified resulting in an impure composition, digested, and then used for absolute quantification of the protein sample.

FIG. 2 depicts another embodiment wherein the expression plasmid is calibrated, digested, added to the sample to be quantified, and then analyzed by mass spectroscopy analysis.

FIG. 3 shows two biological replicates of heterologously overexpressed carousel peptides were compared for reproducibility. A linear regression with a high R² value indicates excellent reproducibility. Each point represents an individual peptide and its peak area from separate overexpression, purification, digestion, and quantitation. Because all peptides were concatenated within the expression plasmid, their abundance should be equivalent unless poor tryptic cleavage efficiency or wall loss occurs.

FIG. 4 is a comparison of carousel peptides with 3 internal standards (Pierce peptides, myoglobin, green fluorescent protein). Two sets of externally calibrated proteins were compared: 90 fmol of each the Pierce peptide mix and myoglobin pure protein in known aliquot volume. The slopes for peak area for the carousel peptide area (synthesized isotope labeled peptides) versus the added reference peptides of the Pierce and myoglobin standards were very similar (2.3989 and 2.2377) indicating the accuracy in protein quantitation. Each point represents a unique peptide (and its corresponding labeled and unlabeled peak area) with multiple peptides from each standard solution being measured.

FIG. 5 is a comparison of the peak area of heterologously produced peptides versus those synthetically produced peptides. Significant variability exists between these two types of peptide abundances, which could be due to the shelf life of the synthetic peptides or loss during freeze/thaw cycles.

### DETAILED DESCRIPTION OF THE INVENTION

The invention features compositions and methods that are useful for absolute quantification of proteins. In some embodiments, the methods described herein produce hundreds of isotopically labeled tryptic peptides simultaneously for quantitative mass proteomic spectrometry for use in environment and biomedical applications. The invention is based, at least in part, on the discovery that generating internal standard peptides (“carousel peptides”) from a superprotein comprising a detectable protein for peptide calibration improves efficiency of internal standard peptide production and accuracy of quantification of the analyte protein.

The invention features a method for making and using the carousel peptides for quantitative proteomics and provides for the production and accurate calibration of isotopically labeled peptides. The calibrated peptides may then be used as standards and calibration standards for a variety of analytical and qualitative purposes. The carousel peptides are prepared using nucleic acid expression constructs which produce a “superprotein” comprising the carousel peptides linked by multiple proteolysis sensitive sites to form a chain of peptides, and a detectable protein or detectable moiety fused to the chain of peptides (also referred to as a “reporter” or “reporter function”). The superprotein may also comprise a purification tag (also referred to as “purification handle” or “tag”). The nucleic acid expression constructs are used to generate a superprotein comprising the concatenated peptides in a protein expression system. The quantity of the superprotein produced, and accordingly the peptides contained therein, is subsequently determined through the quantitative assessment of the reporter function, followed by proteolytic digestion and stable isotope labeling of the peptides. The peptides may then be used in protein quantitation studies.

### Peptide Standards for Absolute Protein Quantification

The production of peptide standards for use in analytical methods has traditionally been expensive, time consuming, and subject to inaccuracies. Traditionally, standards are generally extensively purified in order to precisely determine their concentration. Even so, in many instances, quantitation accuracy remains unreliable due to difficulty in re-dissolving the purified peptides as a result of their chemical characteristics, such as hydrophobicity, peptide size, etc. As a result of using the current peptide standards, there are problems with reproducibility and accuracy, which can lead to significant issues in the determination of protein concentrations because inaccuracies in protein standard concentrations result in inaccuracies in the measurement of protein concentration.

Some practitioners have used highly purified peptides (see Gerber et al., 2003 PNAS) or reduced purity synthetic peptides (Gerber et al., 2003 PNAS 100, 6940-6945; Hilpert K, et al. Nature Protocols 2, 1333-1349 (2007)), often by simultaneous co-synthesis of peptides (U.S. Pat. No. 8,501,421 B2). However, both of these approaches result in the quantitation of peptide standards prior to lyophilization, which results in the calibration of the standards to potentially be inaccurate upon resolubilization. Additionally, since each peptide is custom synthesized in these approaches, large scale production of isotopically labeled peptides is not cost feasible using either of these approaches.

Thus, in some aspects, the invention provides methods for generating internal standard peptides (“carousel peptides”) useful for absolute quantification of an analyte protein. Methods of the invention feature use of a superprotein for generating carousel peptides. The superprotein comprises a plurality of carousel peptides consecutively linked by protease cleavable sites to form a chain of peptides and a detectable protein or detectable moiety fused to the chain of peptides or otherwise disposed within the superprotein. In certain embodiments, the detectable moiety is a fluorescent protein or an enzyme.

The inventive low cost technology described herein solves the problem of accurate calibration of a mixture of many peptide compositions (and hence not a single purified peptide) simultaneously and while in the dissolved phase in the presence of contaminating substances through the use of specifically designed superproteins produced in well-defined protein expression and isotopic labeling systems.

### Superprotein Construct Design

In some aspects, the invention provides a superprotein. The superprotein is useful for generating internal standards used for absolute quantification of an analyte protein. In several embodiments, the superprotein of the invention comprises a plurality of carousel peptides linked consecutively by protease cleavable sites to form a chain of peptides and a detectable protein fused to the chain of peptides. In other embodiments, the superprotein comprises a plurality of carousel peptides linked consecutively by protease cleavable sites to form a chain of peptides and a detectable protein fused within the superprotein separate from the chain of peptides.

The hybrid protein or superprotein gene construct contains a series of nucleic acids encoding the desired amino acid codons for each carousel peptide, assembled to include a protease-cleavable site (i.e., a spacer) separating each peptide. The carousel peptides are selected or pre-determined based on a desired set of proteins found within an organic sample such as a cell, tissue, or whole organism. Each carousel peptide typically represents one single protein (“analyte protein”) of the comparative sample. Upon cleavage of the hybrid protein or superprotein with a protease, each peptide may exist as an individual hybrid protein fragment (“carousel peptide”). In some embodiments, the chain of carousel peptides comprises tryptic biomarker sequences pre-identified from a global proteomic survey of biological samples.

In particular embodiments, a plurality of carousel peptides are generated when the superprotein is cleaved or digested with a protease. In certain embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 40, at least 60, at least 80 carousel peptides are generated. In other embodiments, a carousel peptide may be incorporated more than once within the superprotein to create a unique stoichiometry of said peptide. Thus, in some embodiments, the carousel peptides may uniquely represent at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different analyte proteins. It is envisioned that this method may be employed on a large scale potentially quantifying up to 100,000 proteins. The invention provides for easy, efficient methods for building whole proteome libraries or large scale quantitation of peptides. In some embodiments, the superprotein comprises carousel peptides representing a human proteome. In some other embodiments, the carousel peptides represent a proteome comprising proteins or proteomes of marine organisms. In particular embodiments, the carousel peptides represent a proteome useful for analysis of ocean biochemical health (Saito et al. “Multiple nutrient stresses at intersecting Pacific Ocean biomes detected by protein biomarkers.” Science. 345: 1173-1177; Saito et al. “Needles in the blue sea: Sub-species specificity in targeted protein biomarker analyses within the vast oceanic microbial metaproteome.” Proteomics. 00: 1-11.)

The superprotein further comprises a detectable protein or a detectable moiety. The detectable protein or moiety is fused to the chain of carousel peptides. In particular embodiments, the detectable protein is a fluorescent protein (e.g., enhanced green fluorescent protein (eGFP), red fluorescent protein (RFP), far-red fluorescent protein, blue fluorescent protein (BFP), cyan fluorescent protein (CFP), yellow fluorescent protein (YFP), orange fluorescent protein (OFP)). In some embodiments, the detectable protein may be cleaved from the carousel peptides. In other embodiments, the detectable protein may be cleaved from a position separate from the peptides in the superprotein. In other embodiments, the detectable protein is an enzyme. The detectable protein or detectable moiety is used for quantification of the superprotein or the carousel peptide(s).

In other aspects, the invention provides a polynucleotide encoding the superprotein or the chain of carousel peptides described herein. In still other aspects, the invention provides an expression vector comprising the polynucleotide herein. In some embodiments, the sequence of the polynucleotide encoding the chain of carousel peptides is determined by back-converting tryptic biomarker sequences pre-identified from a global proteomic survey of biological samples into DNA sequences. One aspect of the invention is that the carousel peptides are naturally derived from the protein targets of interest in contrast to commercial peptides which are in vitro synthesized which may impact the accuracy of quantification of proteins in the natural environment.

The polynucleotide encoding the chain of carousel peptides is inserted into an expression system which includes all suitable elements for the expression of the chain of carousel peptides into a hybrid protein (i.e., a fusion protein or “superprotein”). Incorporated with the expression system are additional elements including a promoter, a resistance marker, a purification handle (e.g., tag), and a plurality of restriction digest cloning sites. The expression system may also include a reporter function or polynucleotide encoding a detectable protein or detectable moiety (e.g., a fluorescent protein such as eGFP). If the expression systems includes a reporter, a polynucleotide encoding the chain of carousel peptides may be inserted into the appropriate site in the expression system to link or fuse the chain of peptides to the detectable protein or detectable moeity. Thus, when the construct is expressed, a superprotein comprising a chain of carousel peptides and a detectable protein or moiety fused to the chain of peptides is produced.

The polynucleotide encoding the chain of carousel peptides or the superprotein may be constructed with a plurality of restriction sites for insertion into a range of expression systems and for diagnostic restriction digestion for construct confirmation. In certain embodiments, a polynucleotide encoding the superprotein or the chain of carousel peptides is synthesized. The synthesized hybrid gene sequence or polynucleotide encoding the superprotein may be inserted into the expression vector by ligation.

In some embodiments, the expression system of the hybrid gene or superprotein is adapted for high protein expression such as E. coli, but may be any suitable vector. The bacterial overexpression plasmid may be synthesized. In some other embodiments, the bacterial overexpression plasmid is an E. coli plasmid. The synthesized bacterial overexpression plasmid may comprise a concatenated DNA sequence for the carousel peptides to be labeled, a purification handle, and a reporter region. In one embodiment, the purification handle is a histidine tag or poly(histidine) sequence. In other embodiments, the purification handle is a biotin tag, a myc tag, an HA tag, a FLAG tag, a 3× FLAG tag, a V5 tag, NE tag, chitlin binding protein (CBP) tag, maltose binding protein (MBP) tag, or any other affinity tags or epitope tags as known in the art. In one embodiment, the reporter region or detectable protein comprises an enhanced green fluorescent protein (eGFP) sequence.

### Polypeptide Expression and Purification

Superproteins or carousel peptides of the invention are useful as internal standards (or generating internal standards) for absolute quantification of analyte proteins. Recombinant superproteins of the invention are produced using virtually any method known to the skilled artisan. Typically, recombinant proteins or recombinant polypeptides are produced by transformation of a suitable host cell with all or part of a polypeptide-encoding nucleic acid molecule or fragment thereof in a suitable expression vehicle. Accordingly, the invention provides methods of producing a polypeptide of the invention, the method comprising (a) heterologously expressing an expression vector comprising a polynucleotide encoding the polypeptide in a host cell; and (b) isolating the polypeptide from the host cell (may be optional in some embodiments).

Those skilled in the field of molecular biology will understand that any of a wide variety of expression systems may be used to provide the recombinant polypeptide. The precise host cell used is not critical to the invention. A polypeptide of the invention may be produced in a prokaryotic host (e.g., E. coli, E. coli BL-21) or in a eukaryotic host (e.g., Saccharomyces cerevisiae, insect cells, e.g., Sf21 cells, or mammalian cells, e.g., NIH 3T3, HeLa, COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

In some embodiments, the polypeptides of the invention are produced in a bacterial expression system. One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. In some embodiments, to facilitate purification of the recombinant polypeptide, the polypeptide comprises an epitope tag fused to the polypeptide. The polypeptide is then isolated using an antibody against the epitope tag. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column. In particular embodiments, the purification tag, epitope tag, or sequence tag is a Histidine tag. In certain embodiments, the purification column comprises Ni-NTA Agarose. Once isolated, the recombinant protein can, if desired, be further purified, e.g., by high performance liquid chromatography (see, e.g., Fisher, Laboratory Techniques in Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

In other embodiments, the recombinant protein is not isolated and is used in an impure or contaminated form as a mixture comprising host cell materials, inclusion bodies, reagents, and the like included in the expression of the recombinant protein. In such embodiments, the recombinant polypeptide is expressed and is not further separated from the endogenous proteins present, such as inclusion bodies, in the expression system. The impure recombinant polypeptide mixture may then be calibrated in the same manner as the isolated synthesized proteins described above.

### Protein Detection, Quantification, Peptide Isotope Labeling and Protease Digestion

After isolation or purification (or only expression) of the synthesized protein (superprotein), the synthesized protein is calibrated. The calibration comprises measuring the concentration of the synthesized protein by fluorescence (e.g., eGFP fluorescence) and UV-VIS. Thus, in some embodiments, the methods of the invention comprise the step of measuring fluorescence of the isolated superprotein relative to a fluorescence standard to determine an amount of the superprotein. In other embodiments, the calibration comprises measuring activity of the synthesized superprotein, if the superprotein comprises an enzyme as a reporter. Protein calibration by fluorescence may be performed using a wide range of suitable fluorescence reading devices many of which are compatible with measuring the fluorescence of multiple different samples simultaneously, allowing the calibration of more than one superprotein at the same time. Furthermore, this method provides an economical and time-saving option in comparison to protein calibration via mass spectrometry.

In specific embodiments, the superprotein may be calibrated after protease cleavage. The detectable protein utilized for calibration is generally susceptible to proteolysis; however, genetic engineering or cloning may be employed to alter the inherent proteolysis sites in the detectable protein, making it resistant to proteolysis and therefore unaffected and capable of use in calibration.

After calibration, the superprotein may be cleaved or digested with a protease. Suitable proteases include trypsin, serine proteases, cysteine proteases, metalloproteases, chymotrypsin, thermolysin, pepsin, cathepsin, hepsin, SCCE, TADG12, TADG14, Lys-C, Lys-N, Asp-N, Glu-C, Arg-C, carboxypepidase (A, B, C), and the like. Digestion of the superprotein generates fragments corresponding to the carousel peptides. Thus, in certain embodiments, methods of the invention comprise the step of contacting the isolated superprotein with a protease, thereby generating a carousel peptide.

Optionally, to determine or control for protease cleavage efficiency, fluorescence, or activity of the superprotein may be additionally measured after the superprotein is cleaved with the protease. Cleavage efficiency of the superprotein ultimately impacts the accuracy of quantification of the analyte protein using internal standards generated from cleavage of the superprotein. A cleaved or digested detectable protein (e.g., fluorescent protein) does not generate a detection signal; thus, measurement of fluorescence or activity of the superprotein comprising the detectable protein before and after digestion with a protease enables measurement of cleavage efficiency. Accordingly, in some embodiments, methods of the invention comprise measuring fluorescence or activity of the isolated superprotein before and after it is contacted with the protease relative to a fluorescence or activity standard; and comparing the fluorescence or activity measured before and after protease digestion to determine a cleavage efficiency.

The detectable protein may be flanked with unique restriction enzyme sites or at least sites which do not exist in the region of the construct coding the peptides. This allows the detectable protein to be cleaved out of the construct in case it interferes with peptide measurement.

In some embodiments, the synthesized protein is isotopically labeled. Isotopic labeling of a superprotein or carousel peptide of the invention may be performed by metabolic methods. Metabolic methods for peptide labeling incorporate isotopes present in the culture media supplemented with heavy isotope-labeled amino acids. Accordingly, in some aspects, the invention provides methods comprising the step of culturing a host cell expressing a superprotein or carousel peptides of the invention in a medium, wherein the medium comprises an isotopic label.

Stable isotopes may also be incorporated enzymatically, generally by protease digestion in the presence of ¹⁸O-labled water. Additionally, stable isotopes are incorporated prior to the expression of the superprotein during the overexpression of the recombinant plasmid using a bacterial growth media comprising stable isotopes. Heavy isotopes which may be used include, but are not limited to, ¹³C, ¹⁵N, ¹⁷O, ¹⁸O, ²H, ³⁴S ⁷⁴Se, ⁷⁶Se, ⁷⁸Se, ⁸²Se, or the like. In particular embodiments, the labeling of the synthesized protein comprises the use of H₂¹⁸O buffer and digesting with trypsin. In other embodiments, the labeling comprises the use of ¹⁵N. After labeling, the soluble isotopically labeled peptide can be used by mixing it with samples and then performing the desired analysis.

Accordingly, in some aspects, the invention features a composition comprising an isolated superprotein, a protease (e.g., trypsin), and an isotopic label. In other aspects, the invention provides methods comprising the step of contacting the isolated superprotein and/or cleaved superprotein with an isotopic label. In particular embodiments, the steps of contacting the isolated superprotein with a protease and contacting the isolated superprotein and/or cleaved superprotein with an isotopic label are performed substantially simultaneously.

In other aspects, the present invention includes a spacer region disposed between each of the carousel peptides. The spacer region is typically a sequence unique from the carousel peptides and sensitive to an enzymatic activity (e.g., protease digestion, restriction enzyme digestion). In several embodiments, the spacer region is comprised of a sequence with high sensitivity to protease digestion which results in a highly efficient and/or timely reaction. Furthermore, the spacer region may comprise at least 2 amino acids and is often about 6 amino acids. In other embodiments, the spacer region comprises up to 10, 15, 20, 25, or 30 amino acids.

This design would greatly decrease the cost of peptide production by using an overexpression system, then by fusing the peptides to GFP, or similar fluorescent protein that would be able to be calibrated easily and importantly, calibrated when in solubilized form. Finally by using incorporation of ¹⁸O from H₂¹⁸O during the trypsin digestion isotope incorporation, and/or ¹⁵N and/or ¹³C labeling of cellular material in the overexpression culture media will be uniform and inexpensive. Once a plasmid is constructed, an unlimited supply of peptides can be made at relatively low cost, also resolving the shelf-life problem of chemically synthesized peptides. Each new batch of superprotein generated is then self-calibrated which eliminates inaccuracies due to protein freeze/thaw degradation and maintains reproducibility for subsequent experiments. This approach further solves the cost problems associated with both previous patent approaches. The peptides described herein may be interchangeably referred to herein as “GFP-carousel labeled peptides” or “carousel peptides.”

### Mass Spectroscopy and Analysis

The peptides of the invention are useful for quantification of polypeptides by mass spectrometric methods. Accordingly, in some aspects, the invention provides a method for absolute quantification of a polypeptide by mass spectrometry, the method comprising obtaining a mass spectra of a composition comprising the polypeptides described herein. Mass spectrometric methods and methods for obtaining mass spectra of a sample are known by those skilled in the art.

The low-cost methods of making and using carousel peptides for quantitative proteomics described herein solve the problem of accurate calibration of a mixture of many peptide compositions simultaneously. The methods described herein comprise calibrating the peptides in the dissolved phase, in the presence of contaminating substances, through the use of specifically designed hybrid proteins produced in well-defined protein expression and isotopic labeling systems.

The method of making and using the carousel peptides for quantitative proteomics provides a method for the production and accurate calibration of isotopically labeled peptides. The method described herein results in the ability to precisely determine peptide concentrations without complete purification. The method comprises synthesizing peptides as part of a hybrid protein made from a nucleic acid expression construct in a bacterial expression system. The protein produced from the expression system, in addition to comprising the peptides to be isotopically labeled, is most often concatenated.

### Use of the Hybrid Protein or Superprotein and Labeled Peptides

The synthesized protein also comprises a minimum of one other functional amino acid sequences. The functional amino acid sequence comprises a reporter function (i.e., a detectable moiety, such as a detectable protein). A second functional amino acid sequence comprises a “purification handle.” The hybrid protein or superprotein synthesized by the expression system is often partially purified, most often by utilizing the “purification handle.” The reporter function is then used to precisely determine the amount of hybrid protein or superprotein recovered after the purification step. The method further comprises isotopic labelling of the synthesized superprotein after determination of the superprotein (or carousel peptide) concentration. After isotopically labeling the synthesized superprotein or carousel peptides, it can be used in protein quantitation studies. The synthesized carousel peptides can be quantified by multiple methods in soluble form to avoid re-solubilization issues and can be restocked regularly from the original plasmid. According to one aspect, the superprotein may be digested to produce carousel peptides, and the unpurified reaction may be added directly to a protein sample for protein quantification of said sample.

The method described herein greatly decreases the cost of carousel peptide production by using an overexpression system and by fusing the peptides to green fluorescent protein (“GFP”) or a similar fluorescent protein that would be easily calibrated when in solubilized form. Additionally, ¹⁸O, from H₂¹⁸O, is incorporated into the synthesized superprotein during the trypsin digestion of the superprotein. In one embodiment, ¹⁵N is incorporated into the synthesized superprotein, instead of ¹⁸O. In another embodiment, ¹⁵N and ¹⁸O are incorporated into the synthesized superprotein and/or carousel peptide. In yet another embodiment, ¹³C is incorporated into the synthesized protein and/or carousel peptide, instead of ¹⁸O. In another embodiment, ¹³C and ¹⁸O are incorporated into the synthesized protein and/or carousel peptide. In still another embodiment, ¹⁸O, ¹⁵N, and ¹³C are incorporated into the synthesized protein and/or carousel peptide.

Once a plasmid is constructed, an unlimited supply of carousel peptides can be made at relatively low cost, which resolve shelf-life problems of chemically synthesized peptides.

### Kits

The invention provides kits for generating internal standards (e.g., carousel peptides) useful for absolute quantification of analyte proteins. In one embodiment, the kit includes an expression vector comprising polynucleotides of the invention (e.g., polynucleotides encoding a superprotein comprising a plurality of carousel peptides consecutively linked by a protease cleavable site to form a chain of peptides and a detectable protein fused to the chain of peptides). In some embodiments, the kit comprises a sterile container which contains a composition of the invention; such containers can be boxes, ampoules, bottles, vials, tubes, bags, pouches, blister-packs, or other suitable container forms known in the art. Such containers can be made of plastic, glass, laminated paper, metal foil, or other materials suitable for holding a composition comprising a polynucleotide. In some other embodiments, the kit further includes reagents for manipulation of a polynucleotide or expression vector, expression of the polynucleotide, purification of the polypeptide(s) expressed, digestion or cleavage of polypeptide(s) produced, isotopic labeling of the polypeptide(s), and/or measurement of fluorescence or activity of the detectable polypeptide (e.g., fluorescent or enzymatic activity standards). In still other embodiments, the kit further includes host cells and/or culture media for expression of polynucleotides of the invention.

If desired a composition comprising a polynucleotide and/or polypeptide of the invention (e.g., a polynucleotide encoding a superprotein or a chain of carousel peptides as described herein) is provided together with instructions for producing an internal standard useful for absolute quantification of analyte proteins. The instructions will generally include information about the use of the composition for the absolute quantification of analyte proteins. In other embodiments, the instructions include at least one of the following: description and/or sequences of the polynucleotides, carousel peptides and/or analyte proteins; instructions for storage of the compositions; instructions or protocols for expression of the polynucleotides; instructions or protocols for purification, isotopic labeling, and/or measurement of polypeptides or polypeptide amounts; calibration instructions; mass spectrometric protocols; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Production of Carousel Peptides

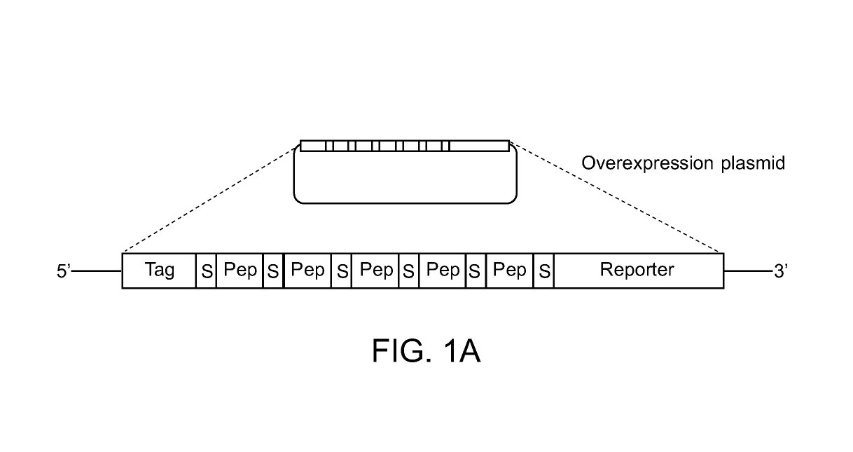

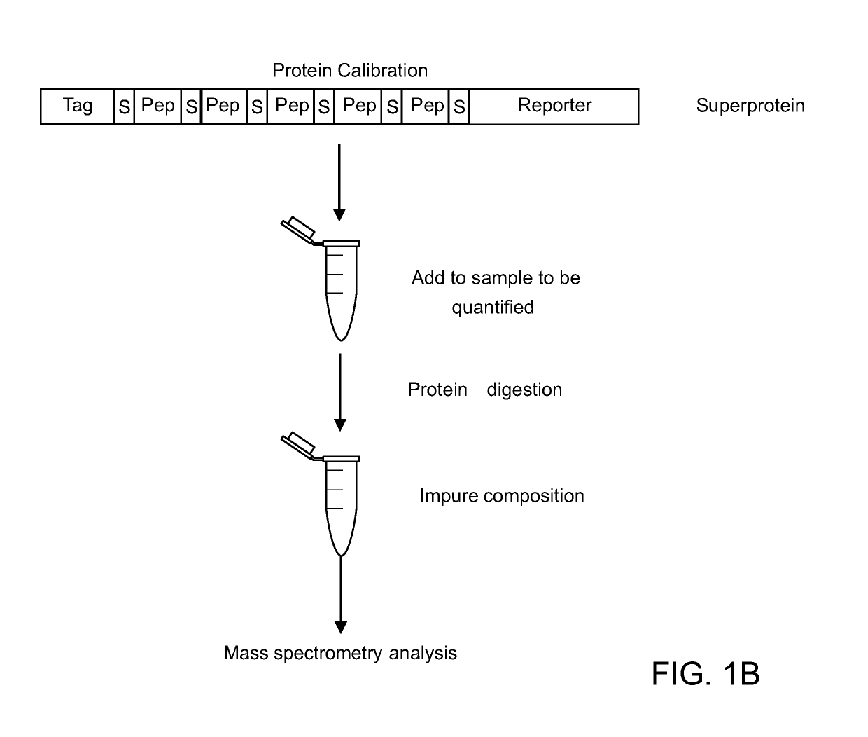

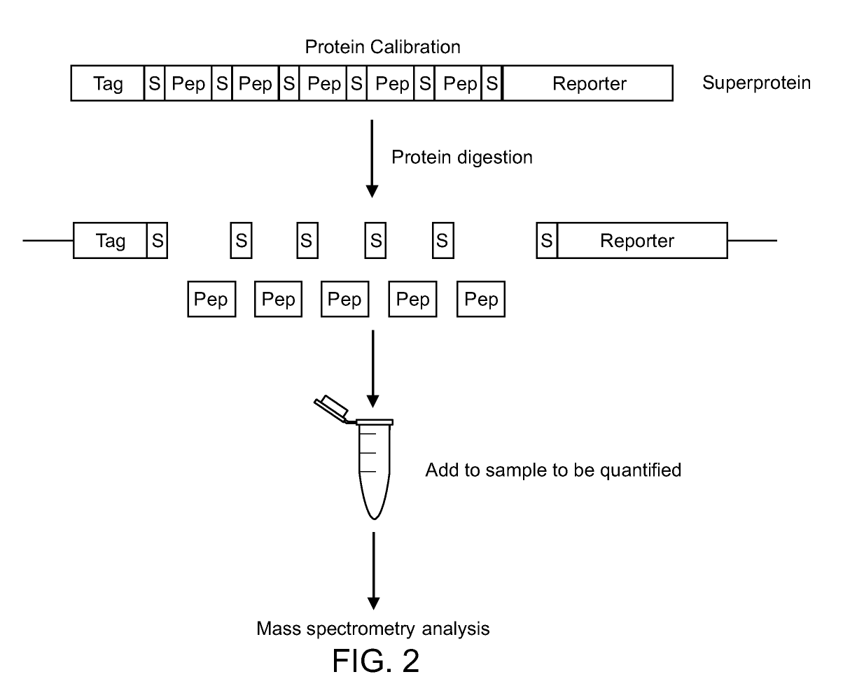

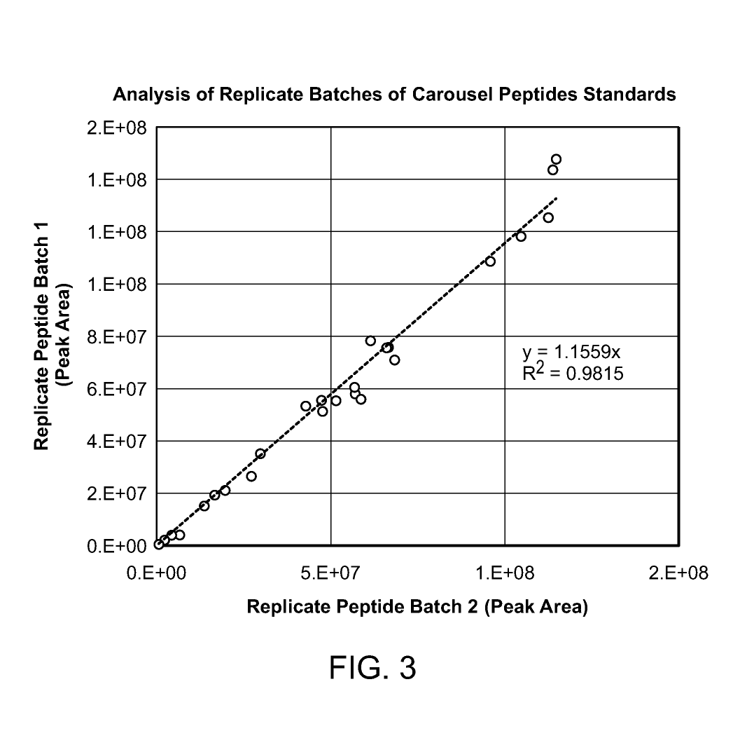

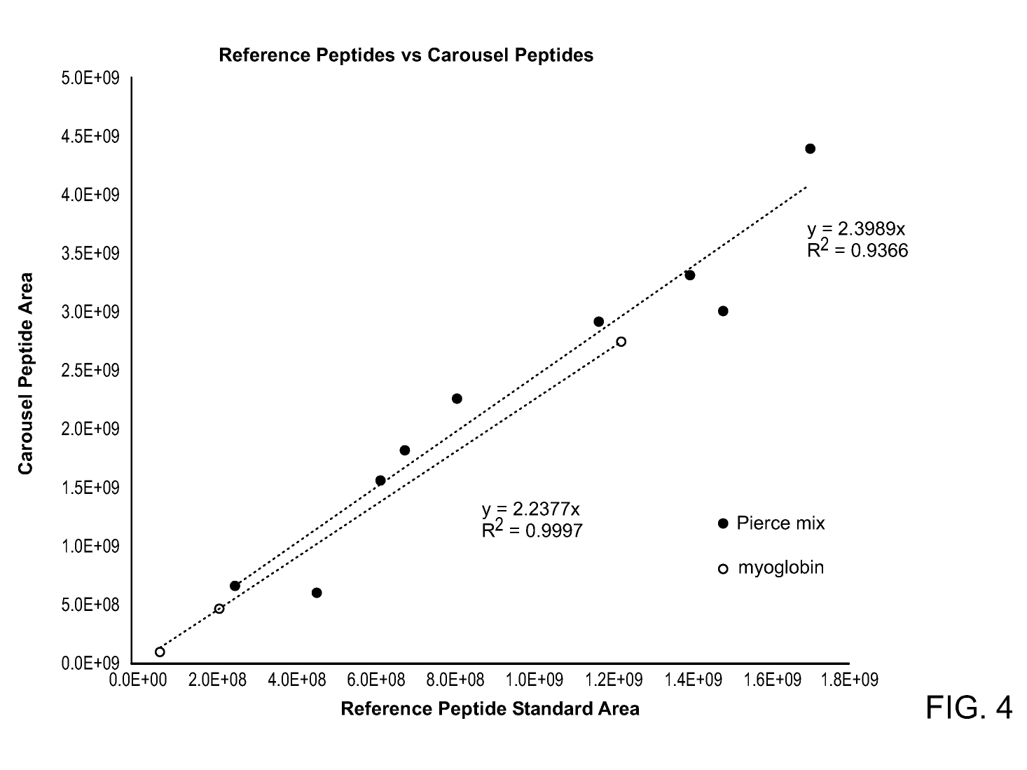

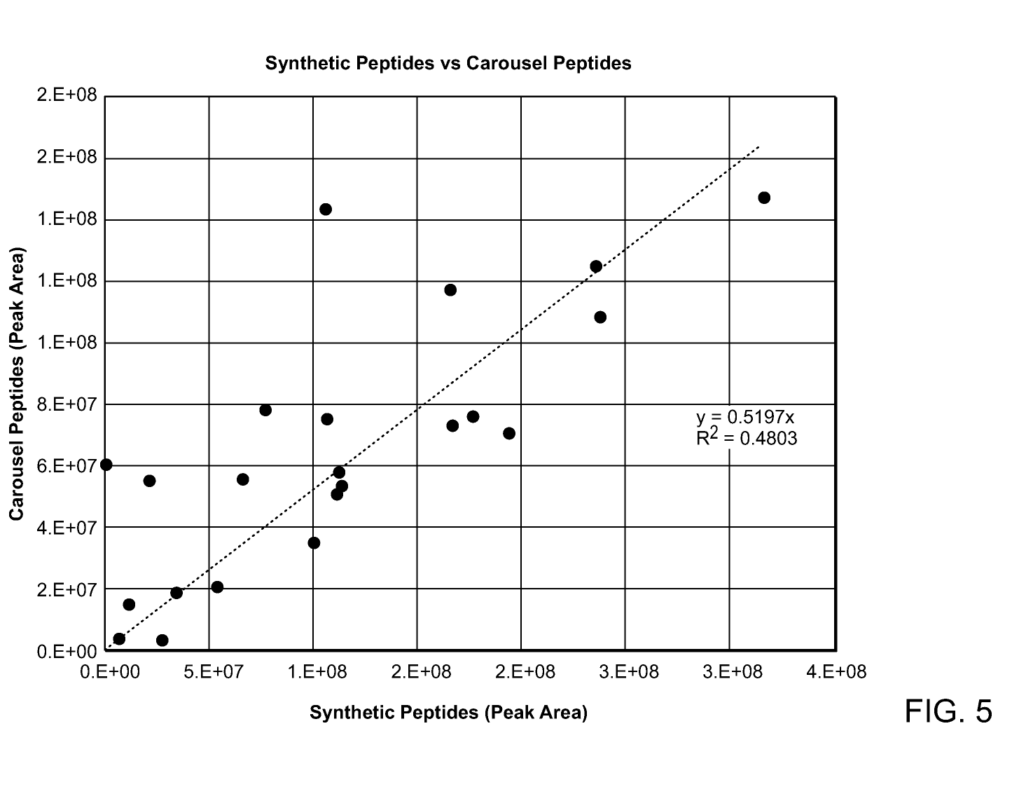

FIG. 1 shows a schematic of the carousel peptide production protocol described herein. Tryptic peptide biomarker sequences were identified from a global proteome survey of biological samples. The tryptic peptide sequences were then back-converted into DNA sequences, concatenated, and fused with the DNA sequence for enhanced green fluorescent protein (eGFP). This DNA sequence was synthesized and ligated within a bacterial overexpression vector with a T7 promoter, kanamycin resistance, and histidine purification tag (Novagen pET-30a). Purified plasmid was transformed into BL21 E. coli cells (Novagen Tuner™ (DE3) pLysS) according to standard protocol and grown on agar plates containing 100 mg/ml Kanamycin at 37° C. overnight. Individual colonies were picked and grown in 1 ml of ¹⁵N labeled culture media (Cambridge Isotope Laboratories Bioexpress Cell Growth Media U-15N, 98%) containing 100 mg/ml Kanamycin for 3 hours at 34° C. Individual colonies were also picked and grown in 1 ml of standard LB media containing 100 mg/ml Kanamycin for 3 hours at 34° C. Both types of cultures were processed in the same way for all remaining steps unless otherwise noted. 500 μl of culture was inoculated into 2×25 ml volumes of corresponding media. Cultures were then induced with 1 mM IPTG (optimized concentration) after 3 hours of growth at 34° C. Induced cells were harvested after 6 hours of growth at 34° C. (growth time previously optimized). Harvested cells were centrifuged at 6500 g for 20 minutes at 4° C. Supernatant was removed and pellets were frozen at −20° C. Pellets were then lysed with 2.5 ml soluble lysis reagent (Novagen BugBuster protein extraction reagent amended with 1 ml (25 units) of Benzonase Nuclease). Inclusion bodies were also purified for the LB grown cells according to the manufacturer's protocol. Lysed extracts were then purified with a Nickel-loaded NTA resin (Novagen His-Bind purification kit) in column mode according to standard protocol. GFP fluorescence of purified protein solutions were measured at an excitation wavelength of 485 nm and emission of 530 nm on a Molecular Devices Spectramax plate reader. Purified proteins were concentrated to a volume of 200 μl and washed 2× with 2 ml of 100 mM ammonium bicarbonate buffer (Ambic) in a Vivaspin 6 ultrafiltration spin column (Sartorius Stedhim). Protein was quantified with a BioRad DC Protein Assay kit according to protocol.

Additional ¹⁸O isotope labeling was conducted on a subset of protein extracts, while others were left with solely 15N labeling to produce three labeled variants of peptide standards: ¹⁵N, ¹⁸O, and ¹⁵N+¹⁸O. ¹⁸O labeling was conducted by trypsin digestion of protein extracts in ¹⁸O—H₂O (Cambridge Isotope Laboratories ¹⁸O water), where all reagents in the ¹⁸O digestions were made up using ¹⁸O water except trypsin and acetic acid. For ¹⁵N only digestions, standard high-purity laboratory water (Fisherbrand Optima LC/MS) was used and both digestions were carried out according to the same protocol as described here. Samples for ¹⁸O labeling were first exchanged in 100 μl of sample with ¹⁸O water three times in an ultrafiltration spin column (Vivaspin 500 Sartorius Stedhim). The ¹⁵N-only sample was exchanged similarly using unlabeled water (Fisherbrand Optima LC/MS) to keep handling of all samples the same. The protein samples were reduced with 5 μl of 200 mM DTT in 100 mM Ambic at 56° C. and 400 rpm for one hour, alkylated with 20 μl of 200 mM iodoacetamide in 100 mM Ambic for 1 hour at 400 rpm RT with an additional 1 hour incubation, at 400 rpm at RT with 20 μl of 200 mM DTT. A 1:50 trypsin:protein digestion was done with 1 mg (in 1 μl of 50 mM acetic acid) of trypsin (Promega) overnight at 37° C., 400 rpm, pH=8.0. Samples were then brought down to a pH of 4.5 with 1 μl of acetic acid (Fluka) and centrifuged at 14,100 g for 30 minutes. Supernatant was removed and put into an ethanol cleaned tube for mass spectrometry analysis. Table 1 provides a summary of the steps for generating the peptide standards described herein.

### Example 2

### An Exemplary Protocol for Production of Carousel Peptides

This example describes one embodiment of the inventive system. Those of ordinary skill in the art understand that various changes and modifications may be made therein without departing from the invention.

### Carousel Peptide Design

Synthetic peptides were created by heterologous overexpression within E. coli BL21 strain. The peptides sequences were selected from discovery proteomic datasets, and reverse translated into corresponding DNA sequences using a web-based tool (http://www.ebi.ac.uk/Tools/st/emboss_backtranseq/). Peptides were chosen with an effort to minimize the presence of methionine and cysteine residues, which can be oxidized and create variability in analyses. Biomarkers for two global nitrogen regulatory proteins were chosen from abundant proteins identified within a metaproteomic discovery dataset. For the purposes of this environmental example, a tryptic peptide from each protein was targeted: the P-II protein (VNSVIDAIAEAAK, MW 1299.70 g/mol) and the NtcA protein (LSHQAIAEAIGSTR, MW 1452.76 g/mol). DNA sequences for target peptides were then concatenated with a 6 amino acid spacer region inserted between each target sequence, and an eGFP (fluorescent protein) sequence added to the 3′ end. The resulting DNA sequence was synthesized with flanking DNA sequence associated with BamH1 and Xho1 (for 5′ and 3′ ends, respectively) and ligated into a PET30a Novagen overexpression plasmid with an enterokinase sequence added to the 3′ end prior to a histidine tag region.

Additional peptides may be engineered into the plasmid as one or more internal references. Bovine serum albumin (BSA) peptides provided an efficient internal standard which included 3 extra peptides integrated into the superprotein corresponding to the BSA protein. The internal standard allows the detectable protein to be removed from the protein if desired and may allow peptide calibration post-digestion.

### Protein Overexpression

The synthetic plasmid was inserted into a Novagen BL21 strain (Tuner™ competent cells), protein expression was induced with IPTG, and the plasmid was harvested at late log growth.

### Python Software for Alternate Codon Usage

The use of the a single 6 amino acid spacer (18 DNA bp) between each target peptide (thus repeated many times within the synthesized DNA) can create significant problems for DNA synthesis and PCR verification. As a result, the codon usage for the spacer region is alternated between target peptide sequences. Codon usage was based on those commonly used by E. coli.

### Fluorescent Protein and Total Protein Quantitation

Peptide calibration was performed prior to peptide digestion by fluorescence measurement which has proven to be highly accurate or at least as accurate as the commercial peptide systems. This method also removed additional mass spectroscopy runs to calibrate the peptide standards. GFP fluorescence of purified protein solutions were measured at an excitation wavelength of 485 nm and emission of 530 nm on a Molecular Devices Spectramax plate reader. GFP fluorescence can be measured simultaneously on a plurality of superproteins by using a multi-well plate in the fluorescent plate reader which increases the ability to employ absolute protein quantification on a larger scale.

### Mass Spectrometry

Protein quantitation was conducted on Thermo Fusion mass spectrometer and analyzed by Skyline software. Mass spectrometry conditions were optimized for each peptide (collision energy and S-lens), and analyzed using chromatographic scheduling to increase the resolution for each peptide. Chromatographic separation was done with a 45 min gradient of 5 to 35% buffer B (where buffer A was 0.1% formic acid in water (Fisher Optima) and buffer B was 0.1% formic acid in acetonitrile (Fisher Optima)) at 4 μL/min. LOD and LOQ were 0.009 fmol and 0.025 fmol for peptide 1 (P-II) and 0.013 fmol and 0.035 fmol for peptide 2 (NtcA), respectively.

### Example 3

### Cleavage Spacer Regions for Production of Carousel Peptides

Shown in Table 1 below are examples of cleavage spacer regions comprising high cleavage efficiency. These amino acid sequence of the spacer region is selected for high sensitivity to proteolysis (in this case high sensitivity to trypsin) and high reproducibility. To overcome problems with synthesis is multiple identical spacer regions within a construct, multiple DNA sequences using varying codons are utilized which all encode the selected amino acid sequence.

TABLE 1
Nucleotide Sequences of the spacer
amino acid sequence TPEFLR

| Seq. | Spacer Combinations |
|---|---|
| 1 | ACTCCTGAATTTCTGCGT |
| 2 | ACCCCCGAATTCCTTCGC |
| 3 | ACTCCTGAATTCCTTCGC |
| 4 | ACCCCCGAATTTCTGCGT |
| 5 | ACTCCCGAATTCCTGCGC |
| 6 | ACCCCTGAGTTTCTTCGT |
| 7 | ACACCAGAATTTCTCCGG |
| 8 | ACACCAGAGTTCCTCCGG |
| 9 | ACACCAGAGTTTCTGCGT |
| 10 | ACTCCTGAATTCCTCCGG |
| 11 | ACACCAGAATTCCTTCGC |
| 12 | ACCCCCGAATTTCTCCGG |

### Example 4

### Carousel Peptides for Absolute Protein Quantification of Protein Targets for Detecting Multiple Nutrient Stresses in the Ocean

Described below in Table 2 is a listing of several protein targets of interest for detecting multiple nutrient stresses in the ocean and their representative carousel peptide for absolute protein quantification. The proteins listed below are involved in the ocean ecosystem including nitrogen regulation, nutrient conditions and stresses, and microbial interactions.

TABLE 2
Protein targets of interest for detecting multiple nutrient
stresses in the ocean and their representative carousel
peptide for absolute protein quantification

| No. | Peptide AA Sequence | Target Protein |
|---|---|---|
| 1 | GTPGDLGAGH-K | PsaB |
| 2 | LLEDDNKVTVSP-R | PsaF |
| 3 | PGISPNF-R | PsaL |
| 4 | DGLTGQATF-R | Psb28 |
| 5 | GVDEPVVPDI-R | Psb28 |
| 6 | ETTETESQNYGY-K | PsbA1 |
| 7 | LAFYDYVGNSPA-K | PsbB |
| 8 | TGEPALDLP-K | PsbB |
| 9 | ITGELYG-R | PsbO |
| 10 | VDNPATFELFGKPGHFD-R | PsbO |
| 11 | LMDAIDAGQPLVLDG-K | PsaA |
| 12 | GYWQELIESIVWAHN-K | PsaA |
| 13 | QILVEPVFAQFVQAASG-K | PsaB |
| 14 | NAAMNEIQIDLGIAF-K | PsaB |
| 15 | SLLAAATWPLAAFGEFTSG-K | PsaF |
| 16 | FVNGTASALEAVYSW-K | Psb28 |
| 17 | ATADGDALTTSTDFEGTY-R | PsbO |
| 18 | FGQEEETYNIVAAHGYFG-R | PsbA1 |
| 19 | QPNIPPADATVENPPADLFT-R | PsaL |
| 20 | VNSVIDAIAEAA-K | P-II (glnB, glnK) |
| 21 | LSHQAIAEAIGST-R | NtcA |
| 22 | MTPGVAFLYGGLA-R | Ammonium transporter |
| 23 | SKLEDDPANPELILTA-R | PhoP |
| 24 | GNDSQEESLMEQI-R | Urease subunit alpha |
| 25 | ITANPAITHGISEHVGTLENG-K | Urease subunit alpha |
| 26 | SLGFHLNISAGTSI-R | Urease |
| 27 | HVQLVEFGGT-K | Urease |
| 28 | NSLEDTTEEQGSLEIP-R | Urease |
| 30 | VEANIGAPQVSYR | Elongation factor EF-2 |
| 31 | ALQALSEEDPTFR | Elongation factor EF-2 |
| 32 | IINEPTAAALAYGLDK | DnaK2 (heat shock protein hsp70-2) |
| 33 | IVNEPTAAALAYGLDK | DnaK2 (heat shock protein hsp70-2) |
| 34 | LVELGAETPGENPYVAEMY-K | NH₄ monooxygenase/CH₄ monooxygenase |
| | | subunit C |
| 35 | TYCYIAQVPGVY-K | Nitrite reductase |
| 36 | TFCYIAEVPGVY-K | Nitrite reductase |
| 37 | FDYDGDYGTVLNR | Sulfolipid (UDP-sulfoquinovose) |
| 38 | cVQLALENPPQ | Sulfolipid (UDP-sulfoquinovose) |
| 39 | NEAVENDLIVDNK | Sulfolipid (UDP-sulfoquinovose) |
| 40 | FDYDGDYGTVLNR | Sulfolipid (UDP-sulfoquinovose) |
| 41 | VASLTGADINYLPNPR | Sulfolipid (UDP-sulfoquinovose) |
| 42 | NEAVENDLIVDNK | Sulfolipid (UDP-sulfoquinovose) |
| 43 | TLDQLLFLYYNK | Sulfolipid (UDP-sulfoquinovose) |
| 44 | LHNFISSAESP-K | Flavodoxin |
| 45 | AGADMVGYVD-K | Flavodoxin |
| 46 | TVGIYYATTTG-K | Flavodoxin |
| 47 | NIFLAQPWNSMDL-K | NirK |
| 48 | TALQGGEVV-K | NirK |
| 49 | QYQSQLLFVPTDEHV-R | NirK |
| 50 | GMGPGGSFELTP-K | Ribonucleoside-diphosphate reductase |
| 51 | SGGGVGINYSEL-R | Ribonucleoside-diphosphate reductase |
| 52 | TSFSIHDSSLEAHLVAS-R | Ribonucleoside-diphosphate reductase |
| 53 | GFISYSGNPR | Cobalamin synthesis protein/P47K |
| 54 | TDLVSDDALDTLESR | Cobalamin synthesis protein/P47K |
| 55 | DKVPVTILTGFLGSGK | Cobalamin synthesis protein/P47K |
| 56 | GYWETSDENIEQLQ | Cobaltochelatase |
| 57 | DLNLIVSELEAcHR | RUBISCO small chain |
| 58 | DLNLVVSELEAcHR | RUBISCO small chain |
| 59 | FDSLINSADNVMTYK | Glutamine synthetase |
| 60 | EGYFPVSPNDTAQDIR | Glutamine synthetase |
| 61 | HAPSFLAFTNPTTNSYK | Glutamine synthetase |
| 62 | VNIDQAK | Ni-Superoxide dismutase |
| 63 | AEELMAAVEK | Ni-Superoxide dismutase |
| 64 | DTAAQETEHAFAHFR | Rubrerythrin |
| 65 | DSGAEAEFAEQSSESK | Rubrerythrin |
| 66 | GAVLLLDEIDLASNK | CobS |
| 67 | FVGTNILNEAFLER | CobS |
| 68 | ESYTPEK | CobS |
| 69 | QSFLDLYTK | CobS |
| 70 | GYISPYFATDTER | Chaperonin Cpn60/TCP-1 |
| 71 | TGKPLVIIAEDIEK | Chaperonin Cpn60/TCP-1 |
| 72 | IAENAGSNGAVIAENVK | Chaperonin Cpn60/TCP-1 |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A superprotein useful for generating an internal standard for quantifying an analyte protein, the superprotein comprising:
(a) a plurality of carousel peptides consecutively linked by a protease cleavable site to form a chain of peptides, wherein each carousel peptide is a fragment of an analyte protein pre-identified as a product of protease cleavage of the analyte protein; and
(b) a detectable protein fused to the chain of peptides.

2. A composition for generating an internal standard for quantifying an analyte protein, the composition comprising:
(a) the superprotein of claim 1;
(b) an isotopic label; and
(c) a protease.

3. The superprotein of claim 1, wherein a purification tag is fused to a 3′ end or 5′ end of the superprotein.

4. The superprotein of claim 1, wherein the superprotein comprises at least 2, at least 3, at least 4, at least 5, at least 10, at least 20, at least 30, at least 40, at least 50, at least 60, at least 70, at least 80, or at least 100 carousel peptides.

5. The superprotein of claim 1, wherein the carousel peptides are fragments of at least 2, at least 3, at least 4, at least 5, at least 10, at least 20, at least 30, at least 40, at least 50, at least 60, at least 70, at least 80, or at least 100 different analyte proteins.

6. The superprotein of claim 1, wherein the analyte protein is selected from the group consisting of proteins involved in the ocean ecosystem.

7. The superprotein of claim 1, wherein the detectable protein or detectable moiety is a fluorescent protein or an enzyme.

8. The superprotein of claim 1, wherein the fluorescent protein is an enhanced green fluorescent protein (eGFP), red fluorescent protein (RFP), far-red fluorescent protein, blue fluorescent protein (BFP), cyan fluorescent protein (CFP), yellow fluorescent protein (YFP), or orange fluorescent protein.

9. The superprotein of claim 1, wherein the enzyme is contacted with a substrate to produce a detectable signal.

10. The superprotein of claim 1, wherein the protease is trypsin, chymotrypsin, thermolysin, pepsin, a serine protease, a cysteine protease, a metalloprotease, Lys-C, Lys-N, Asp-N, Glu-C, or Arg-C.

11. The superprotein of claim 1, wherein the purification tag is a histidine tag, a biotin tag, myc tag, a hemagglutinin (HA) tag, or a FLAG tag.

12. The composition of claim 2, wherein the isotopic label is selected from the group consisting of ¹³C, ¹⁵N, ¹⁸O, ²H, ³⁴S, ⁷⁴Se, ⁷⁶Se, ⁷⁸Se, and ⁸²Se.

13. An isolated polynucleotide encoding a superprotein of claim 1.

14. An expression vector comprising the polynucleotide of claim 1;
and
(b) a detectable protein fused to the chain of peptides.

15-24. (canceled)

25. A host cell for expressing a superprotein useful for generating an internal standard for quantifying an analyte protein, the host cell comprising the isolated polynucleotide of claim 13.

26. (canceled)

27. A kit comprising the expression vector of claim 14.

28. (canceled)

29. A method for generating a superprotein useful for quantifying an analyte protein, the method comprising:
(a) culturing a host cell in a medium, wherein the host cell heterologously expresses a superprotein useful for generating an internal standard for quantifying an analyte protein, the superprotein comprising
a plurality of carousel peptides consecutively linked by a protease cleavable site to form a chain of peptides, wherein each carousel peptide is a fragment of an analyte protein pre-identified as a product of protease cleavage of the analyte protein; and
a detectable protein fused to the chain of peptides; and
(b) isolating the superprotein.

30-32. (canceled)

33. A method for generating an internal standard useful for quantifying an analyte protein, the method comprising:
(a) culturing a host cell in a medium, wherein the host cell heterologously expresses a superprotein useful for generating an internal standard for quantifying an analyte protein, the superprotein comprising
a plurality of carousel peptides consecutively linked by a protease cleavable site to form a chain of peptides, wherein each carousel peptide is a fragment of an analyte protein pre-identified as a product of protease cleavage of the analyte protein; and
a detectable protein fused to the chain of peptides;
(b) isolating the superprotein;
(c) measuring fluorescence or activity of the isolated superprotein relative to a fluorescence or activity standard to determine an amount of the superprotein; and
(d) contacting the isolated superprotein with a protease, thereby cleaving the superprotein;
(e) contacting the isolated superprotein and/or cleaved superprotein with an isotopic label, thereby generating a carousel peptide useful as an internal standard for quantifying an analyte protein.

34-48. (canceled)

49. A composition for absolute quantification of an analyte protein, the composition comprising an amount of a carousel peptide, an amount of an isolated superprotein, or an amount of a cleaved superprotein, wherein the carousel peptide, isolated superprotein, or cleaved superprotein is generated according to the method of claim 29.

50-51. (canceled)

52. A method for absolute quantification of a polypeptide by mass spectrometry, the method comprising obtaining a mass spectra of the composition of claim 49.

53-56. (canceled)
